SEQUENCE LISTING (40 sequences)

SEQ ID NO: 1 - DNA, 20 bp, Artificial Sequence
  Primer sequence
aacagcgacggaggtctcta

SEQ ID NO: 2 - DNA, 20 bp, Artificial Sequence
  A synthetic primer sequence
ttctcttgtcccgcagactt

SEQ ID NO: 3 - DNA, 26 bp, Artificial Sequence
  A synthetic primer sequence
cagtccgtcaccctggtgtgcgatgc

SEQ ID NO: 4 - DNA, 27 bp, Artificial Sequence
  Primer sequence
cagagtctggggtcacctccagatagc

SEQ ID NO: 5 - DNA, 21 bp, Artificial Sequence
  A synthetic primer sequence
cttggggccctgggcctccga

SEQ ID NO: 6 - DNA, 25 bp, Artificial Sequence
  A synthetic primer sequence
gccttcctgcagtggtacacgggcg

SEQ ID NO: 7 - DNA, 20 bp, Artificial Sequence
  A synthetic primer sequence
tgacttctcctgcatgcact

SEQ ID NO: 8 - DNA, 20 bp, Artificial Sequence
  A synthetic primer sequence
agccggcacctgttgtgcaa

SEQ ID NO: 9 - DNA, 20 bp, Artificial Sequence
  A synthetic primer sequence
tccaaaccagaaaacggaag

SEQ ID NO: 10 - DNA, 20 bp, Artificial Sequence
  A synthetic primer sequence
ctgtgcccgtagtgagatga

SEQ ID NO: 11 - DNA, 23 bp, Artificial Sequence
  A synthetic primer sequence
tgtatcgcaggcactcaggtcag

SEQ ID NO: 12 - DNA, 19 bp, Artificial Sequence
  A synthetic primer sequence
aagtctggtcacggggaat

SEQ ID NO: 13 - DNA, 20 bp, Artificial Sequence
  A synthetic primer sequence
gtcctgctaggaggcgcgag

SEQ ID NO: 14 - DNA, 19 bp, Artificial Sequence
  A synthetic primer sequence
gttctccagatgttcttcg

SEQ ID NO: 15 - DNA, 20 bp, Artificial Sequence
  A synthetic primer sequence
tgcctcagaaagagaaccag

SEQ ID NO: 16 - DNA, 20 bp, Artificial Sequence
  A synthetic primer sequence
atggcaggatgaacaaacac

SEQ ID NO: 17 - DNA, 19 bp, Artificial Sequence
  A synthetic primer sequence
tgcaggactcggtggactt

SEQ ID NO: 18 - DNA, 19 bp, Artificial Sequence
  A synthetic primer sequence
tggactcctgctttgcctg

SEQ ID NO: 19 - DNA, 20 bp, Artificial Sequence
  A synthetic primer sequence
acgctgaggaatggttcaag

SEQ ID NO: 20 - DNA, 20 bp, Artificial Sequence
  A synthetic primer sequence
tagacgcctcaatggtttcc

SEQ ID NO: 21 - DNA, 20 bp, Artificial Sequence
  A synthetic primer sequence
gctcgtcgtcgacaacggct

SEQ ID NO: 22 - DNA, 25 bp, Artificial Sequence
  A synthetic primer sequence
caaacatgatctgggtcatcttctc

SEQ ID NO: 23 - DNA, 25 bp, Artificial Sequence
  A synthetic primer sequence
cctcctccatggatctgcttattca

SEQ ID NO: 24 - DNA, 26 bp, Artificial Sequence
  A synthetic primer sequence
caggtcttcacctgtttgtagctgag

SEQ ID NO: 25 - DNA, 18 bp, Artificial Sequence
  A synthetic primer sequence
cccccggcggcaatagca

SEQ ID NO: 26 - DNA, 20 bp, Artificial Sequence
  A synthetic primer sequence
tcggcgccggggagatacat

SEQ ID NO: 27 - DNA, 23 bp, Artificial Sequence
  A synthetic primer sequence
gcgtacgcaaattaaagtccaga

SEQ ID NO: 28 - DNA, 25 bp, Artificial Sequence
  A synthetic primer sequence
cagcatcctaaacagctcgcagaat

SEQ ID NO: 29 - DNA, 24 bp, Artificial Sequence
  A synthetic primer sequence
gcccgcttctcttacagtgtgatt

SEQ ID NO: 30 - DNA, 25 bp, Artificial Sequence
  A synthetic primer sequence
agtacgtgcagacggtggtagttct

SEQ ID NO: 31 - DNA, 20 bp, Artificial Sequence
  A synthetic primer sequence
agctatgcccggacctccat

SEQ ID NO: 32 - DNA, 20 bp, Artificial Sequence
  A synthetic primer sequence
gcctgcagcaggaggatctt

SEQ ID NO: 33 - DNA, 19 bp, Artificial Sequence
  A synthetic primer sequence
gacaagcagcctgtcaagg

SEQ ID NO: 34 - DNA, 19 bp, Artificial Sequence
  A synthetic primer sequence
accatccagcgtgcgttcc

SEQ ID NO: 35 - DNA, 23 bp, Artificial Sequence
  A synthetic primer sequence
gccacatctaatctcatttcaca

SEQ ID NO: 36 - DNA, 20 bp, Artificial Sequence
  A synthetic primer sequence
ctgggtaacagcagatgcaa

SEQ ID NO: 37 - DNA, 22 bp, Artificial Sequence
  A synthetic primer sequence
gggcgcctggtcaccagggctg

SEQ ID NO: 38 - DNA, 21 bp, Artificial Sequence
  A synthetic primer sequence
ggggccatccacagtcttctg

SEQ ID NO: 39 - DNA, 20 bp, Artificial Sequence
  A synthetic primer sequence
cgaccatctgccgctttgag

SEQ ID NO: 40 - DNA, 20 bp, Artificial Sequence
  A synthetic primer sequence
ccccctgtcccccattccta

(19) United States
(12) Patent Application Publication
(10) Pub. No.: US 2010/0047906 A1
(43) Pub. Date: Feb. 25, 2010

(54) GERM LINEAGE DERIVED FEEDER CELLS AND METHODS THEREOF

(75) Inventors: Satish Totey, Bangalore (IN); Kumar Uday Kulkarni, Bangalore (IN); Shobhit Saxena, Bangalore (IN)

Correspondence Address: SCHWEGMAN, LUNDBERG & WOESSNER, P.A., P.O. BOX 2938, MINNEAPOLIS, MN, 55402, US

(73) Assignee: Stempeutics Research Private Limited, Bangalore (IN)

(21) Appl. No.: 12/488,150

(22) Filed: Jun. 19, 2009

Related U.S. Application Data
(63) Continuation of application No. PCT/IN2007/000593, filed on Dec. 17, 2007.

(30) Foreign Application Priority Data
Dec. 19, 2006 (IN) 2360/CHE/2006

(51) Int. Cl. C12N 5/071

(52) U.S. Cl. 435/366

(57) ABSTRACT

The present disclosure relates to human germ layer derived feeder cells (GLDF cells) and method of generation thereof. Further, it relates to a method for culturing and propagating human embryonic stem cells (hESCs) in a substantially undifferentiated state for several passages on the human GLDF cells. In particular, the present disclosure relates to human GLDF cells which are capable of supporting proliferation of hESCs in a substantially undifferentiated and pluripotent state for several passages.

### PRIORITY

This application is a continuation under 35 U.S.C. §111(a) of international application No. PCT/IN2007/000593, filed Dec. 17, 2007 and published in English as WO 2008/075377 A2 on Jun. 26, 2008, which application claims priority from Indian application serial No. 2360/CHE/2006 filed Dec. 19, 2006, which applications and publication are herein incorporated by reference.

### FIELD OF INVENTION

The present invention relates to Germ Lineage Derived Feeder cells (GLDF cells) for derivation, culture and propagation of human embryonic stem cells (hESCs) in an undifferentiated state.

### BACKGROUND OF INVENTION

Stem cells have the ability to divide without limit and to give rise to specialized cells. They are best described in the context of normal human development. Following the rule according to which, in ontogenesis, the younger the cell, the more pluripotent it is. It has been generally believed that embryonic stem cells are the only truly totipotent cells, whereas adult stem cells are capable of only maintaining the homeostasis of the tissue in which they belong. Embryonic stem cells are uncommitted totipotent cells isolated from embryonic tissue. When injected into embryos, they can give rise to all somatic lineages upon differentiation and give rise to a wide variety of cell types, derived from ectoderm, mesoderm, and endoderm embryonic germ layers. Embryonic stem cells (ESCs) have been isolated from the blastocyst, inner cell mass or gonadal ridges of mouse, rabbit, rat, pig, sheep, primate and human embryos (Evans and Kauffman, 1981; Iannaccone et al., 1994; Graves and Moreadith, 1993; Martin, 1981; Notarianni et al., 1991; Thomson, et al., 1995; Thomson, et al., 1998; Shamblott, et al., 1998, Heins, et al 2004). hESC lines were first isolated by Thomson et al. 1998. These cells have the potential to produce any type of cells of the body in an unlimited quantity and can be genetically altered (Brivanulou et al. 2003).

Currently practiced ESCs culturing methods are mainly based on the use of feeder cell layers which secrete factors needed for stem cell proliferation, while at the same time, inhibit their differentiation. To date, the most commonly used feeder cells are mouse embryonic fibroblasts (MEF) (Thomson et al., 1998; Reubinoff et al., 2000), which are prepared from day 13.5 post-coitum embryos of pregnant mice. However, concerns arise that contaminations, such as rodent viruses or proteins introduced by MEF, may make hESCs unsuitable for therapeutic purposes. Alternative culture systems have therefore been invented to avoid the use of MEF.

Recently, some groups demonstrated that it is possible to culture hESCs on feeder cells that originate from human source (Richards et al., 2003; Amit et al., 2003; Cheng et al., 2003; Hovatta et al., 2003; Lee et al., 2005). Human feeders support prolonged undifferentiated growth of embryonic stem cells. However, the major disadvantage of using human embryonic fibroblasts or adult fallopian tube epithelial cells as feeder cells is that both of these cell lines have a limited passage capacity of only 8-10 times, thereby limiting the ability of a prolonged ES growth period. For a prolonged culturing period, the ES cells must be grown on human feeder cells originated from several subjects which results in an increased variability in culture conditions.

The other systems use a feeder-free environment that cultures hESCs in special media supplemented with Matrigel matrix plus MEF-conditioned medium (Xu et al 2001), fibronectin plus transforming growth factor β1 and basic fibroblast growth factor (bFGF) (Amit et al., 2004), or Matrigel in combination with activator of WNT pathway (Sato et al., 2004), respectively. Moreover, the stable and long-term culture of hESCs and the maintenance of their undifferentiated state still requires feeder cells along with the additional exogenous basic fibroblast growth factor (bFGF) (Kim et al., 2005).

Reubinoff et al (Nat. Biotechnol. 18: 399-404 and Science 282: 1145-7; Reubinoff B E, Pera M F, Fong C, Trounson A, Bongso A. (2000)) reports the derivation of embryonic stem cell lines from human blastocysts. Further, ES cells can be cultured on MEF under serum-free conditions using serum replacement supplemented with basic fibroblast growth factor (bFGF) (Amit M, Carpenter M K, Inokuna M S, Chiu C P, Harris C P, Waknitz M A, Itskovitz-Eldor J, Thomson J A. (2000). Clonally derived human embryonic stem cell lines maintain pluripotency and proliferative potential for prolonged periods of culture. Dev. Biol. 227: 271-8). Under these conditions the cloning efficiency of ES cells is 4 times higher than under fetal bovine serum.

Human ES cells can be cultured on human foreskin feeder layer as disclosed in U.S. patent application Ser. No. 10/368,045. US patent application 20060051862 discloses a method of establishing a feeder cells-free human embryonic stem cell line capable of being maintained in an undifferentiated, pluripotent and proliferative state.

### SUMMARY OF THE INVENTION

The present disclosure relates to the human Germ Lineage Derived Feeder Cells (GLDF cells) and process of its generation. Further, it relates to a method for culturing and propagating human Embryonic Stem Cells (hESCs) in a substantially undifferentiated state for several passages on the GLDF cells. The ability to grow hESCs without differentiation has important applications for therapeutic uses of ESCs for treating human disorders using tissue transplantation and/or gene therapy techniques. In particular, the present disclosure relates to human GLDF cells which are capable of supporting proliferation of hESCs in a substantially undifferentiated and pluripotent state for several passages. This disclosure further relates to a method of generating human GLDF cells.

An aspect of the present disclosure is to provide a method of generating human GLDF cells, comprising culturing hESCs on growth medium to obtain cells of germ lineages; culturing the cells of germ lineages on a GLDF medium comprising of KO-DMEM, growth factors, serum supplement, media supplements or a combination thereof to obtain fibroblast like cells; and treating fibroblast like cells to generate human GLDF cells.

In another aspect the disclosure provides a GLDF medium for generation of the human GLDF cells.

In yet another aspect the disclosure provides human GLDF cells for derivation, culturing and propagation of hESCs in undifferentiated and pluripotent state.

In yet another aspect the disclosure provides human germ lineage derived feeder cells that support the hESC lines in a long term in vitro culture systems.

### BRIEF DESCRIPTION OF DRAWINGS

FIG. 1 is photomicrographs showing (a) day 8 embryoid bodies that were cultured for the derivation of GLDF cells (b) morphology of human GLDF cells at day 1 (c) morphology of human GLDF cells at day 3 and (d) morphology of human GLDF cells at day 5

FIG. 2 shows RT-PCR results showing the expression of differentiation markers on human GLDF cells. Expression of Nestin-220 bp, NF-L-560 bp, βIII tubulin-174 bp, NCAM-757 bp, GATA2-244 bp, GATA4-187 bp, BMP2-328 bp, BMP4-339 bp, HAND1-274 bp, β-actin-353b was screened at Passage 5 (P5)—Lane 1, Passage 10 (P10)—Lane 2, Passage 15 (P15)—Lane 3, Passage 20 (P20)—Lane 4, Passage 25 (P25)—Lane 5.

FIG. 3 shows RT-PCR results showing the expression of pluripotent markers on the human GLDF cells. Expression of Oct4-573 bp, Nanog-262 bp, Sox2-448 bp, Rex1-303 bp, TDGF1-498 bp was screened at Passage 5 (P5)—Lane 1, Passage 10 (P10)—Lane 2, Passage 15 (P15)—Lane 3, Passage 20 (P20)—Lane 4, Passage 25 (P25)—Lane 5. β-actin-353 bp was used as housekeeping control.

FIG. 4 shows RT-PCR results showing the expression of fibroblast markers on human GLDF cells. Expression of Vimentin and P4Hβ was screened at Passage 5 (P5)—Lane 1, Passage 10 (P10)—Lane 2, Passage 15 (P15)—Lane 3, Passage 20 (P20)—Lane 4, Passage 25 (P25)—Lane 5. β-actin-353 bp was used as house keeping control.

FIG. 5 shows high expression of basic FGF in GLDF cells at Passage 5 (P5)—Lane 1, Passage 10 (P10)—Lane 2, Passage 15 (P15)—Lane 3, Passage 20 (P20)— Lane 4, Passage 25 (P25).

FIG. 6 shows photomicrographs showing expression of fibroblast markers using immunocytochemistry was screened at Passage 5 (P5), Passage 10 (P10), Passage 15 (P15), Passage 20 (P20) and Passage 25 (P25). Pictures (a)-(d) shows expression of Vimentin, Pictures (e)-(h) shows the expression of Nestin and Pictures (i)-(l) shows the expression of P4H β.

FIG. 7 shows expression of cell surface markers analyzed by flow cytometry at passage-5 (P5), passage-10 (P10), passage-15 (P15), passage-20 (P20) and passage-25 (P25). The markers used for expression profiling of cell surface markers were CD 50, CD 106, CD 44, CD 54, CD 31, CD 105, CD 90, CD 73, CD 34, CD 45, CD 117, and CD 135.

FIG. 8 shows photomicrograph showing morphology of human embryonic stem cells HUES-7 on GLDF cells at Passage 10 (P10) and Passage 20 (P20).

FIG. 9 shows morphology of human embryonic stem cell line HUES-9 cultured on GLDF feeder cells at Passage 10 (P10) and Passage 20 (P20).

FIG. 10 shows RT-PCR results showing expression of pluripotent markers of human embryonic stem cells HUES-7 cultured on GLDF cells. Expression of Oct 4, Nanog, Sox 2, Rex 1, TDGF 1 and TERT was checked at Passage 5 (P5)—Lane 1, Passage 10 (P10)—Lane 2, Passage 15 (P15)—Lane 3, Passage 20 (P20)—Lane 4.

FIG. 11 shows photomicrograph showing expression of embryonic stem cell markers by immunocytochemistry on human embryonic stem cells HUES-7 cultured on GLDF cells. Expression of Alkaline phosphatase at 20× magnification, OCT-4 at 20× magnification, SSEA-4 at 20× magnification and TRA-1-60 at 20× magnification was checked at Passage 20 (P20).

FIG. 12 shows RT-PCR results showing expression of pluripotent markers of human embryonic stem cells HUES-7 cultured on GLDF cells. Expression of Oct 4, Nanog, Sox 2, Rex 1, TDGF 1 and TERT was screened at Passage 5 (P5)—Lane 1, Passage 10 (P10)—Lane 2, Passage 15 (P15)—Lane 3, Passage 20 (P20)—Lane 4.

FIG. 13 shows photomicrograph showing expression of embryonic stem cell markers by immunocytochemistry on human embryonic stem cells HUES-9 cultured on GLDF cells. Expression of Alkaline phosphatase at 20× magnification, OCT-4 at 20× magnification, SSEA-4 at 20× magnification and TRA-1-60 at 20× magnification was screened at Passage 20 (P20).

### DETAILED DESCRIPTION OF THE INVENTION

The present disclosure relates to human germ layer derived feeder cells (GLDF cells) and process of its generation thereof. Further, it relates to a method for culturing and propagating human embryonic stem cells (hESCs) in a substantially undifferentiated state for several passages on the human GLDF cells. The ability to grow hESCs without differentiation has important applications for therapeutic uses of ESCs for treating human disorders using tissue transplantation and/or gene therapy techniques. In particular, the present disclosure relates to human GLDF cells which are capable of supporting proliferation of hESCs in a substantially undifferentiated and pluripotent state for several passages. This disclosure further relates to a method of generating human GLDF cells.

Unless specifically stated, the terms used in the specification have the same meaning as used in the art. The materials, methods, and examples are illustrative only and not intended to be limiting the scope of the disclosure.

One embodiment of the present invention relates to a method of generating human germ lineage derived feeder cells (GLDF cells), said method comprising:
a. culturing human embryonic stem cells (hESC) on growth medium to obtain germ lineage cells;
b. culturing said germ lineage cells on GLDF medium to obtain fibroblast like cells; and
c. treating said fibroblast like cells to generate human GLDF cells.

An embodiment of the disclosure relates to a method of generating human GLDF cells comprising of culturing hESCs on growth medium to obtain cells of germ lineage; culturing the cells of germ lineage on a GLDF medium comprising KO-DMEM and supplements to obtain fibroblast-like cells; and treating the fibroblast-like cells to generate human GLDF cells.

Another embodiment of the disclosure provides a method of generating human GLDF cells, wherein the hESCs are cultured on growth medium in order to generate embryoid bodies made up of cells of germ lineages. The growth medium comprises of about 80% knockout Dulbecco's minimum essential medium (KO-DMEM) supplemented with about 20% human serum, about 1-2% non-essential amino acid, beta-mercaptoethanol, about 200 mM L-glutamine, and pen-strep.

Yet another embodiment of the disclosure relates to a method of generating human GLDF cells, wherein the embryoid bodies are cultured and passaged on GLDF medium comprising of about 90% KO-DMEM supplemented with about 10% KO-Serum, about 1 mM Gulamine, about 1×10⁻⁸ M dexamethasone, about 1× insulin-transferrin-selenium and about 10 ng/ml epidermal growth factor.

Still another embodiment of the disclosure provides a method of generating human GLDF cells, wherein the growth factors in GLDF medium are selected from a group consisting of about 1-20 ηg/ml transforming growth factor-β-1 (TGF-β-1); epidermal growth factor (EGF; e.g., about 1-20 ng/ml), about 1-20 ng/ml brain derived neurotrophic factor (BDNF), about 1-20 ηg/ml platelet derived growth factor (PDGF), Insulin, selenite, transferrin, about 5-100 ng/ml Activin-A, about 5-100 ng/ml Activin-B, about 1-20 ng/ml Acidic FGF (fibroblast growth factor), about 2-20 ng/ml human Insulin growth factor (IGF), about 10-50 ng/ml Keratenocyte growth factor (KGF), about 5-20 ng/ml stem cell factor (SCF), about 5-20 ng/ml bone morphogenic protein (BMP4), about 10-20 ng/ml hepatocyte growth factor (HGF), about 20-100 ng/ml nerve growth factor (NGF) and a combination thereof for growth medium.

Further embodiment of the present invention relates to a method of generating human GLDF cells, wherein the fibroblast-like cells in step c were treated with mitomycin.

Another preferred embodiment of the disclosure provides human GLDF cells for culturing hESCs, wherein GLDF cells are prepared by the methods as described in the present disclosure.

Yet another embodiment of the disclosure suggests that the human GLDF cells are fibroblast-like cells.

Another embodiment of the disclosure provides human GLDF cells which are negative for the expression of pluripotent markers selected from the group consisting of NANOG, REX-1, TDGF, SOX-2, and TERT.

Yet another embodiment of the disclosure provides human GLDF cells which are positive for the fibroblast phenotypic marker P4HB and mesenchymal stem cell marker Vimentin.

The human GLDF cells of the disclosure shows mesenchymal stem cell phenotypes.

Further embodiment of the disclosure provides human GLDF cells which are positive for mesenchymal stem cell markers CD73, CD105, CD90, and CD44.

The human GLDF cells provided in the disclosure are positive for the expression of differentiation markers selected from the group of NCAM, β-III tubulin, GATA2, Hand1, BMP4, Vimentin, CK18, Nestin, NF heavy chain, GFAP, and CK19.

Still further embodiment of the present disclosure relates to the GLDF medium further comprises serum supplement. The serum supplement comprises serum and/or serum replacement. Further, the serum supplement is provided at the concentration of about 10-30%, preferably about 20%.

Stem cells should be derived and maintained in an undifferentiated state for their use in tissue regeneration. Also they should remain proliferative for long term in-vitro cultures. A significant challenge to the use of hESCs for therapy is that they are cultured in a xeno-free condition on a layer of feeder cells to prevent differentiation. The term ‘Xeno-free’ as used herein refers to cell cultures free from any contamination from animal source other than cells of human origin, wherein the contamination may comprise virus and/or proteins and/or any entity from animal cells other than cells of human origin.

Further, these feeder layers should fully meet the requirements to enable to derive and propagate human embryonic stem cell lines and to control differentiation of stem cells into particular type of tissue required for treatment of each patient.

The present disclosure also provides human feeder cells for the derivation of new hESC lines and culturing in xeno-free conditions. The role of germ lineages derived feeder cells is to support the hESCs in vitro culture systems for long term. The system described in this disclosure allows for proliferation of stem cells for use in studying the biology of stem cell differentiation, and the production of important products for use in human therapy. In particular, the disclosure relates to a method of generating human GLDF cells and human GLDF cells generated thereof. The cells of the present disclosure are capable of supporting derivation of new hESC lines and its proliferation in a substantially undifferentiated state for several passages.

In accordance with the present disclosure the method for generating human GLDF cells comprises preparing a suspension of cells from an undifferentiated hESC culture to generate embryoid bodies which comprises cells of three main germ lineages i.e endoderm, ectoderm and mesoderm. Further, the embryoid bodies are directly plated onto the solid surface of the bio-coated Petri dishes.

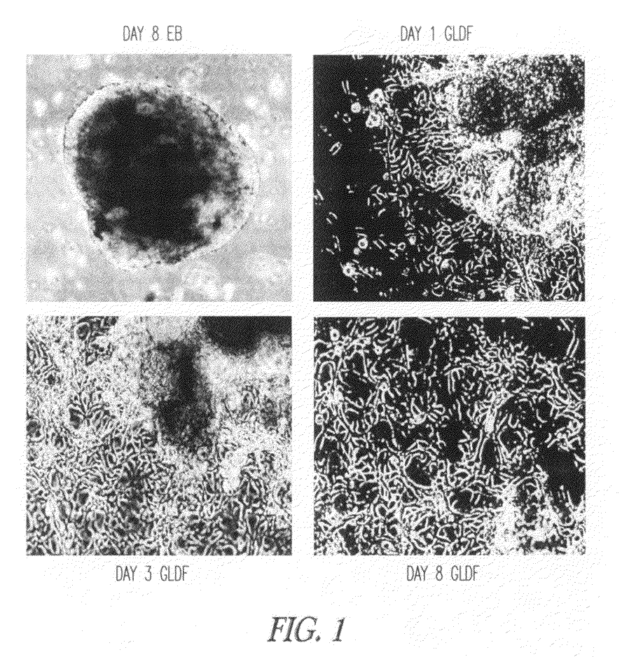

In particular aspect, the present disclosure relates to bio-coating of the Petri dishes with about 0.1% gelatin or about 5 μg/ml collagen IV coating or about 5 μg/ml laminin coating or about 5 μg/ml fibronectin coating or a combination thereof. The embryoid bodies are passaged several times on GLDF medium until they differentiate into fibroblast like cells, herein termed as human GLDF cells. FIG. 1 shows the photomicrographs of day 8 embryoid bodies that were cultured for the derivation of GLDF cells and also the morphology of human GLDF cells at day 1, day 3 and day 5. Detailed procedure of generation of human GLDF cells is provided in Example 1.

Feeder cells described in this disclosure are derived from hESCs that differentiated into mesoderm lineages and are similar to that of mesenchymal stem cells. These cells have high telomerase activity and of embryonic origin. The feeder cells derived by the method provided in the present disclosure secrete all the necessary growth factors such as basic fibroblast growth factor that are required for the derivation of new hESC lines and maintaining it without any differentiation. These feeder cells can be grown and used indefinitely without any limitation of passages and have no batch to batch variations.

Surprisingly, it was found that the human embryonic stem cells (hESCs) can be grown for prolonged period maintaining the undifferentiated and proliferative state of the hESCs without any variability if cultured on germ linage derived feeder cells (GLDF) as disclosed.

The most commonly used feeder cells are mouse embryonic fibroblasts (MEF). However there is a risk of contaminations such as rodent viruses or proteins introduced by MEF which makes the hESc unsuitable for therapeutic use. Currently practiced hESCs culturing methods are mainly based on the use of feeder cell layers which secrete factors needed for stem cell proliferation, while at the same time inhibit their differentiation. The major disadvantage in using the feeder layer cells obtained from human source such as human embryonic fibroblast or adult fallopian tube epithelial cells hESCs is the limited passage capacity of only 8-10 times, thereby limiting the prolonged growth period. The feeder free environment for culturing the hESCs in special media is reported in the prior art but long term culture and maintenance in undifferentiated condition of the hESCs still requires the feeder cells along with the additional exogenous basic fibroblast growth factor. This problem has been solved by the present invention by providing germ lineage derived feeder cells (GLDF) for the prolonged growth of hESCs in undifferentiated state. The human GLDF cells of the present invention are capable of supporting proliferation of the hESCs in undifferentiated state without any contamination for prolonged period.

A preferred embodiment of the present disclosure provides human GLDF cells prepared by the method as disclosed in Example 1, wherein the GLDF cells are fibroblast like cells and are similar to cells of mesenchymal origin derived from ESC lines of human origin. Further, the GLDF cells are capable of forming a mono-layer in the cell culture. Feeder cells disclosed in the present disclosure secretes high amount of growth factors and shows high telomerase activity and hence can be used indefinitely without any limitations.

The human GLDF cells described in the present disclosure are capable of supporting the growth and propagation of hESCs in a long term in vitro culture systems, wherein the stem cells are maintained in substantially undifferentiated and proliferative state.

Expression profile of human GLDF cells for various pluripotent and differentiation markers can be carried out by employing different methods known in the art.

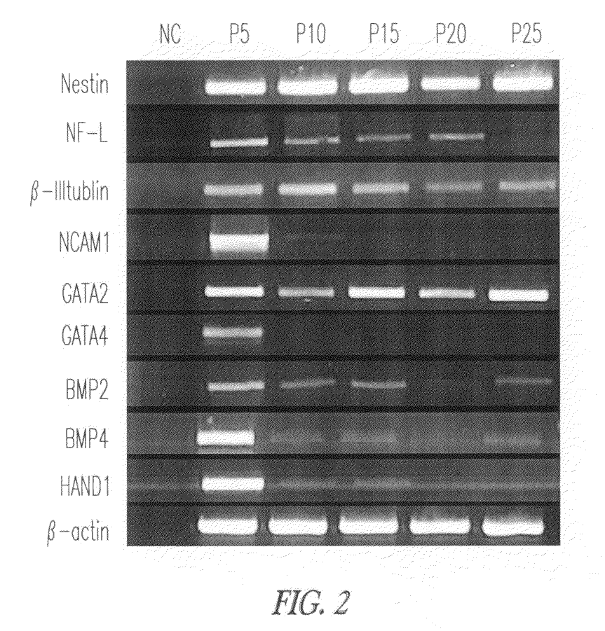

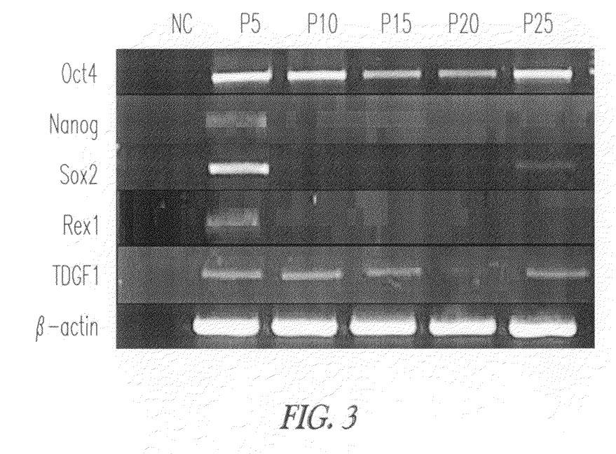

The RT-PCR method was employed for analyzing expression profile of various differentiation markers such as Nestin, NCAM, β-III tubulin, GATA2, GATA-4, BMP2, BMP4, Hand1, Vimentin, NF light chain and GFAP (See Table 1). FIG. 2 shows the RT-PCR results showing the expression of Nestin-220 bp, NF-L-560 bp, βIII tubulin-174 bp, NCAM-757 bp, GATA2-244 bp, GATA4-187 bp, BMP2-328 bp, BMP4-339 bp, HAND1-274 bp at different passages wherein β-actin-353b was used as house keeping control. Upstream and downstream primers were used to screen the expression of various markers as below:

| | |
|---|---|
| Nestin | |
| SEQ ID: 1 - | AACAGCGACGGAGGTCTCTA |
| SEQ ID: 2 - | TTCTCTTGTCCCGCAGACTT |
| NCAM | |
| SEQ ID: 3 - | CAGTCCGTCACCCTGGTGTGCGATGC |
| SEQ ID: 4 - | CAGAGTCTGGGGTCACCTCCAGATAGC |
| β-III tubulin | |
| SEQ ID: 5 - | CTTGGGGCCCTGGGCCTCCGA |
| SEQ ID: 6 - | GCCTTCCTGCAGTGGTACACGGGCG |
| GATA2 | |
| SEQ ID: 7 - | TGACTTCTCCTGCATGCACT |
| SEQ ID: 8 - | AGCCGGCACCTGTTGTGCAA |
| GATA4 | |
| SEQ ID: 9 - | TCCAAACCAGAAAACGGAAG |
| SEQ ID: 10 - | CTGTGCCCGTAGTGAGATGA |
| BMP2 | |
| SEQ ID: 11 - | TGTATCGCAGGCACTCAGGTCAG |
| SEQ ID: 12 - | AAGTCTGGTCACGGGGAAT |
| BMP4 | |
| SEQ ID: 13 - | GTCCTGCTAGGAGGCGCGAG |
| SEQ ID: 14 - | GTTCTCCAGATGTTCTTCG |
| Hand 1 | |
| SEQ ID: 15 - | 5′-TGCCTCAGAAAGAGAACCAG |
| SEQ ID: 16 - | 5′-ATGGCAGGATGAACAAACAC |
| Vimentin | |
| SEQ ID: 17 - | TGCAGGACTCGGTGGACTT |
| SEQ ID: 18 - | TGGACTCCTGCTTTGCCTG |
| NF light chain | |
| SEQ ID: 19 - | ACGCTGAGGAATGGTTCAAG |
| SEQ ID: 20 - | TAGACGCCTCAATGGTTTCC |
| β-actin | |
| SEQ ID: 21 - | GCTCGTCGTCGACAACGGCT |
| SEQ ID: 22 - | CAAACATGATCTGGGTCATCTTCTC |

Human GLDF cells however, do not express any pluripotent markers. The expression of various pluripotent markers was checked by performing RT-PCR. The cells were found negative for the expression of pluripotent markers such as NANOG, SOX-2, REX-1, TDGF-1 and TERT (See Table: 2). FIG. 3 shows RT-PCR results for the expression of Nanog-262 bp, Sox2-448 bp, Rex1-303 bp, TDGF1-498 bp at different passages, wherein Beta-actin marker gene was used as a house keeping control. Upstream and downstream primer sequences for expression of Beta-actin marker gene are as shown in SEQ ID NO.: 21 and SEQ ID NO.: 22. The primer sequences used to screen the expression of various markers are as below:

| | |
|---|---|
| NANOG | |
| SEQ ID: - 23 | CCTCCTCCATGGATCTGCTTATTCA |
| SEQ ID: - 24 | CAGGTCTTCACCTGTTTGTAGCTGAG |
| SOX-2 | |
| SEQ ID: - 25 | CCCCCGGCGGCAATAGCA |
| SEQ ID: - 26 | TCGGCGCCGGGGAGATACAT |
| REX-1 | |
| SEQ ID: - 27 | GCGTACGCAAATTAAAGTCCAGA |
| SEQ ID: - 28 | CAGCATCCTAAACAGCTCGCAGAAT |
| TDGF-1 | |
| SEQ ID: - 29 | GCCCGCTTCTCTTACAGTGTGATT |
| SEQ ID: - 30 | AGTACGTGCAGACGGTGGTAGTTCT |
| TERT | |
| SEQ ID: - 31 | AGCTATGCCCGGACCTCCAT |
| SEQ ID: - 32 | GCCTGCAGCAGGAGGATCTT |

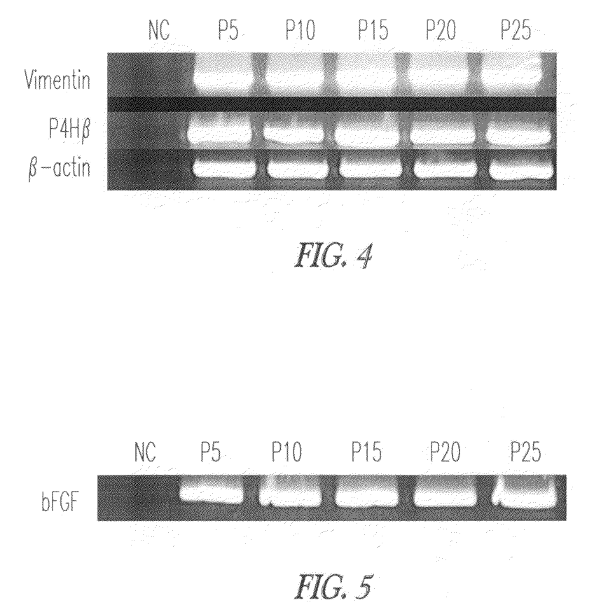

The human GLDF cells disclosed are also found to be positive for the expression of fibroblastic phenotypes as checked by RT-PCR (See Table 3). FIG. 4 shows RT-PCR results for the expression of P4Hβ and the main intermediate filament protein-Vimentin at different passages. β-actin-353 bp was used as house keeping control, the expression of which was brought about by using primer sequences as shown in SEQ ID NO.: 21 and SEQ ID NO.: 22. Primer sequences for Vimentin are as shown in SEQ ID NO.: 17 and SEQ ID NO.: 18. Further the upstream and downstream primer sequences for P4Hβ are as shown below:

| | |
|---|---|
| P4Hβ | |
| SEQ ID NO.: 33 - | GACAAGCAGCCTGTCAAGG |
| SEQ ID NO.: 34 - | ACCATCCAGCGTGCGTTCC |

The expression of basic Fibroblast Growth Factor (bFGF) by human GLDF cells was separately checked at passage 5, 10, 15, 20 and 25 employing RT-PCR (See FIG. 5), wherein the primer sequences used are as shown below:

| | |
|---|---|
| bFGF | |
| SEQ ID NO.: 35 - | GCCACATCTAATCTCATTTCACA |
| SEQ ID NO.: 36 - | CTGGGTAACAGCAGATGCAA |

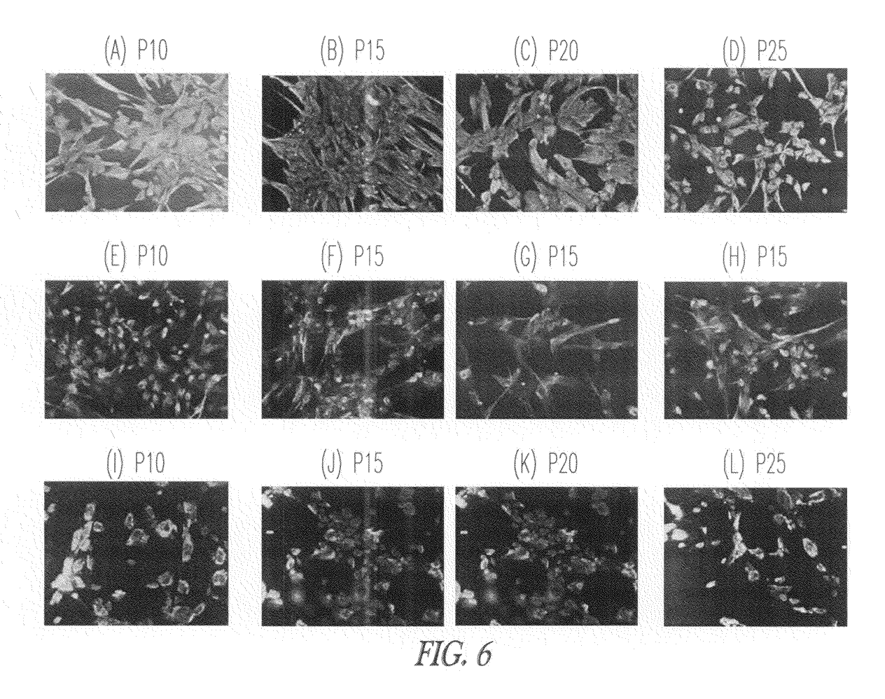

The expression of various fibroblast markers on human GLDF cells was also found positive as checked by immunocytochemistry. FIG. 6 shows photomicrographs for the expression of Vimentin, Nestin and P4H β. Immunocytochemistry was carried out after different passages in order to study the up-regulation and down-regulation of the genes.

The expression profiling of differentiation markers was again performed using RT-PCR. The expression of markers specific for Ectoderm cell lineages, Endoderm cell lineages and Mesoderm cell lineages was checked and were found positive for lineage specific markers.

In accordance with the present disclosure the human GLDF cells are further characterized for Ectoderm markers and were found positive for markers selected from the group consisting of NCAM and beta III tubulin, nestin, MAP2.

In accordance with the present disclosure the human GLDF cells are characterized for Endoderm markers and were found positive for markers selected from the group consisting of GATA2, FLk1, alpha actinin.

In accordance with the present disclosure the human GLDF cells are characterized for mesoderm markers and were found positive for markers selected from the group consisting of Hand1, BMP4, Brachyury, Hnf4, Hnf beta, Foxa2

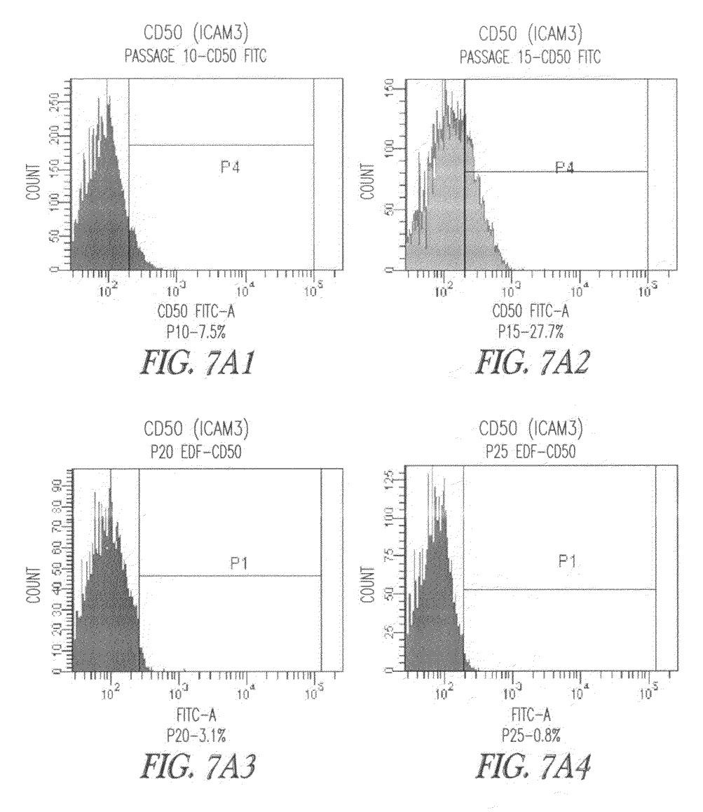

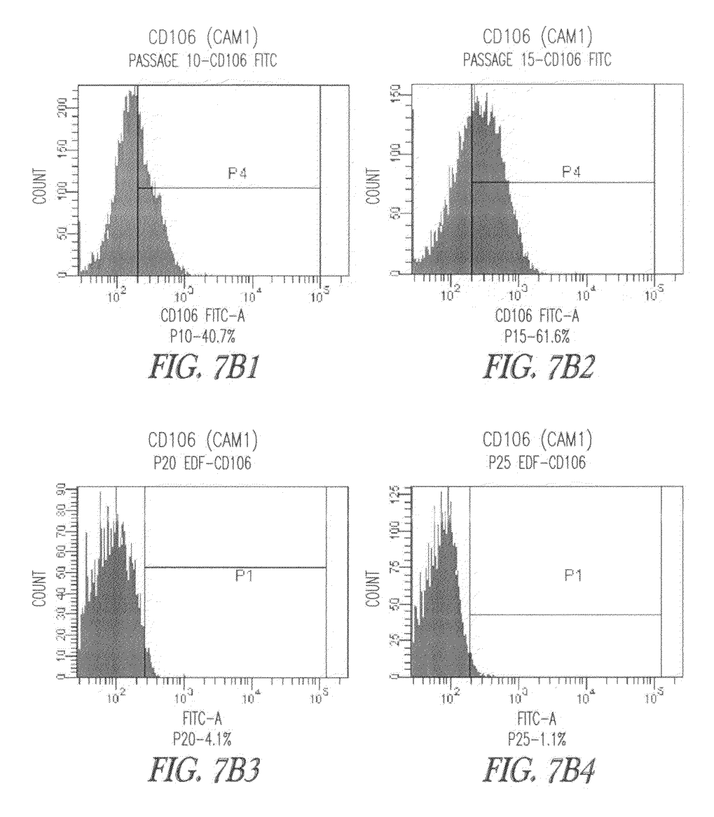

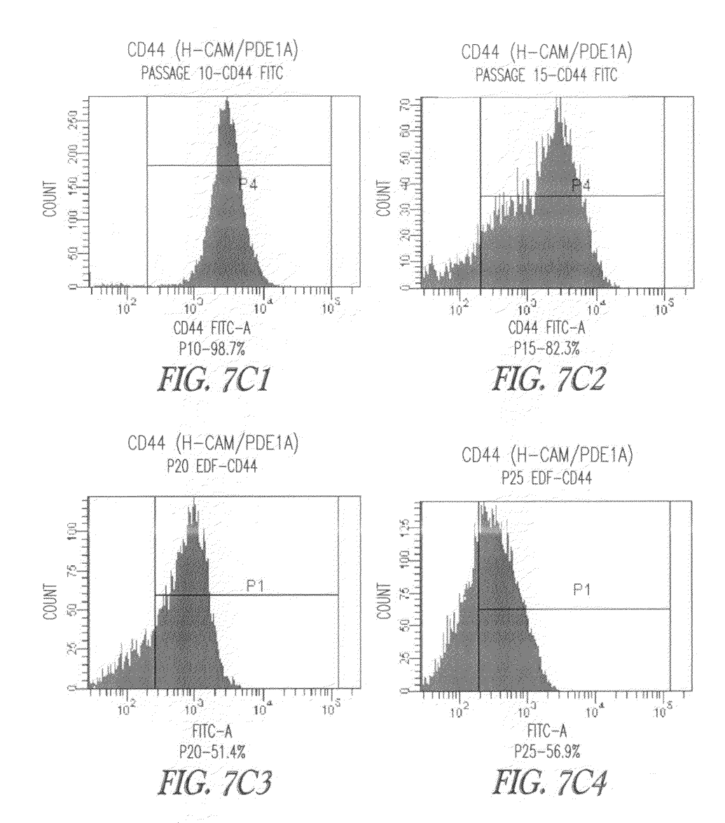

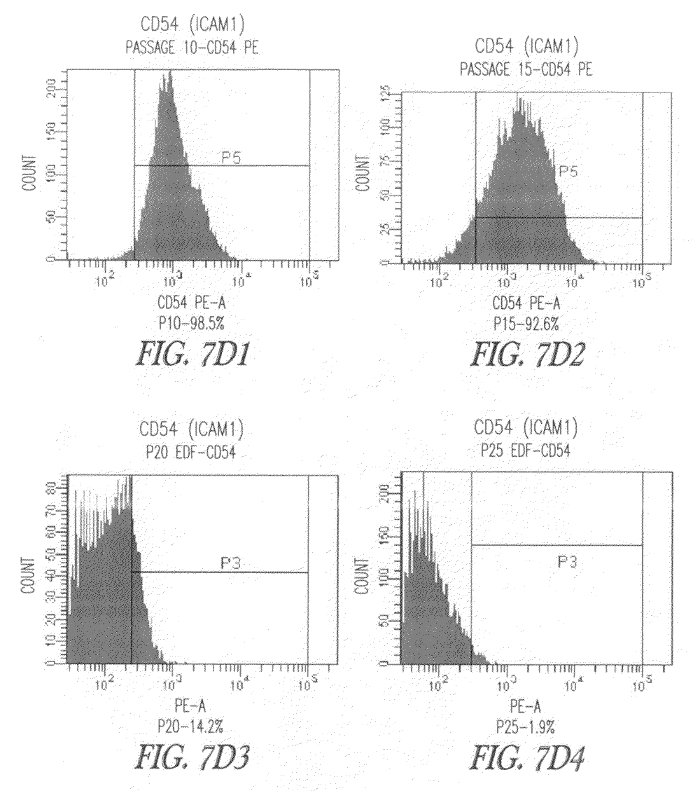

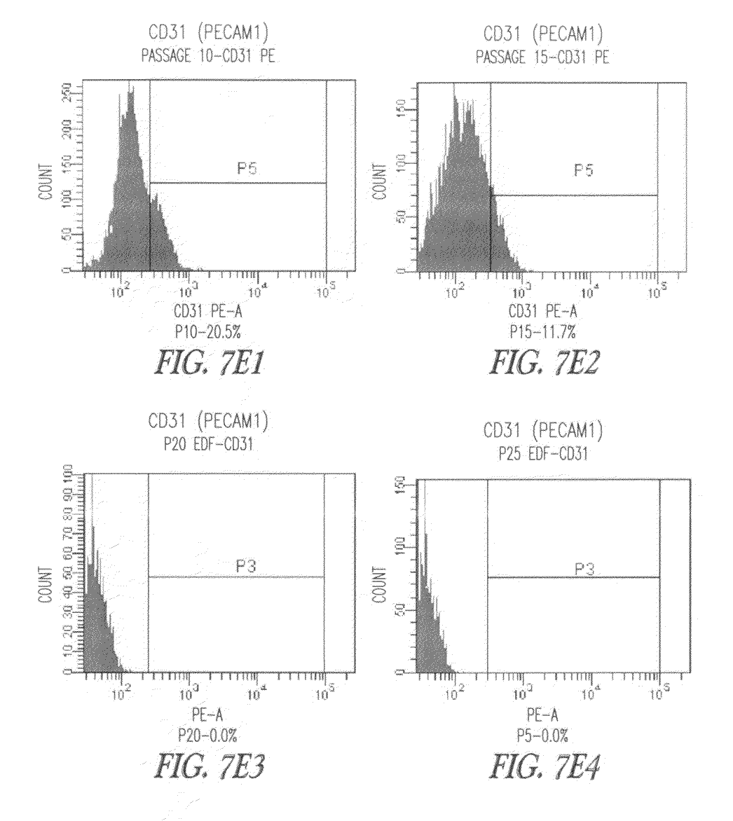

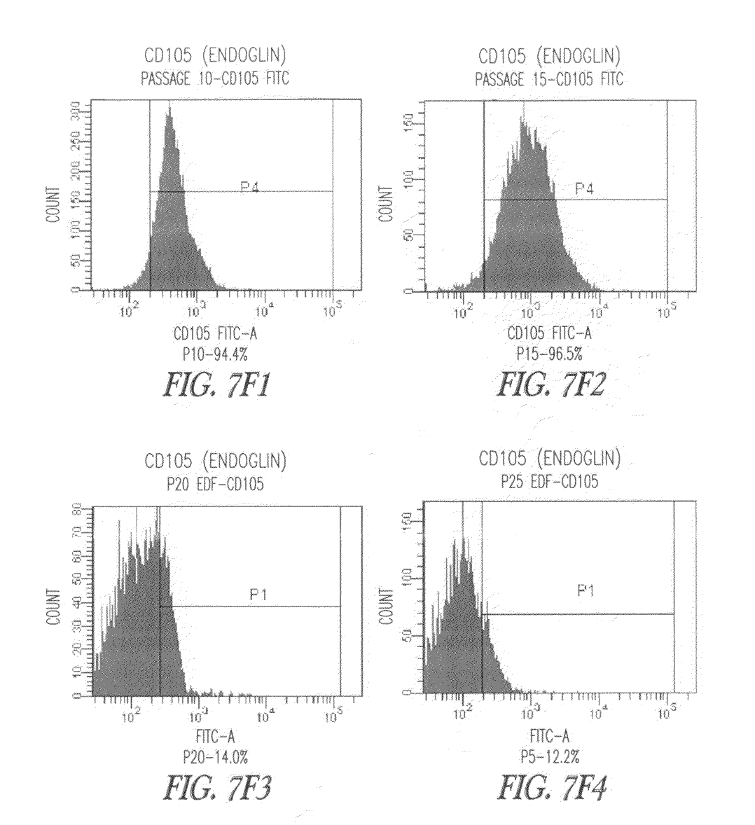

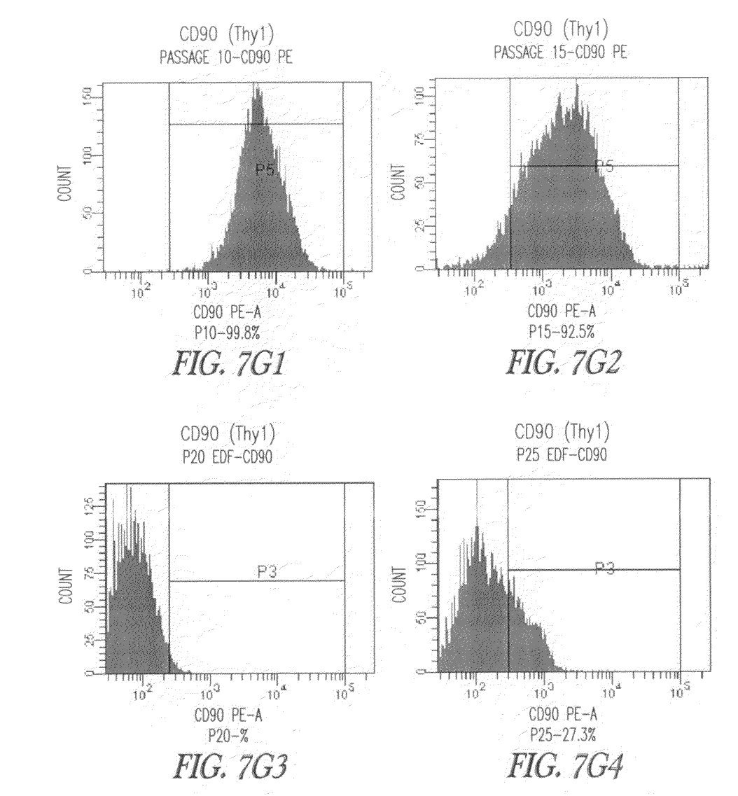

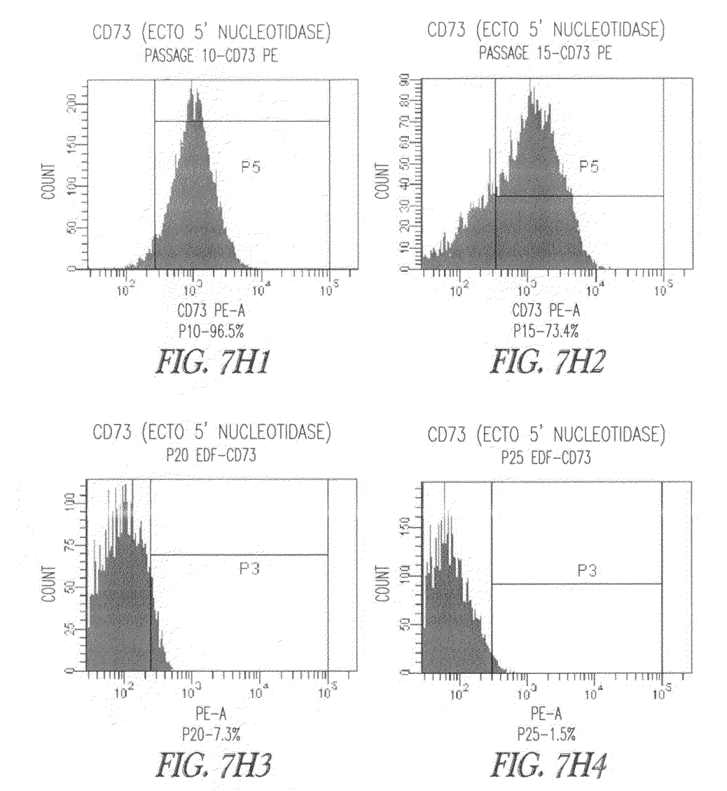

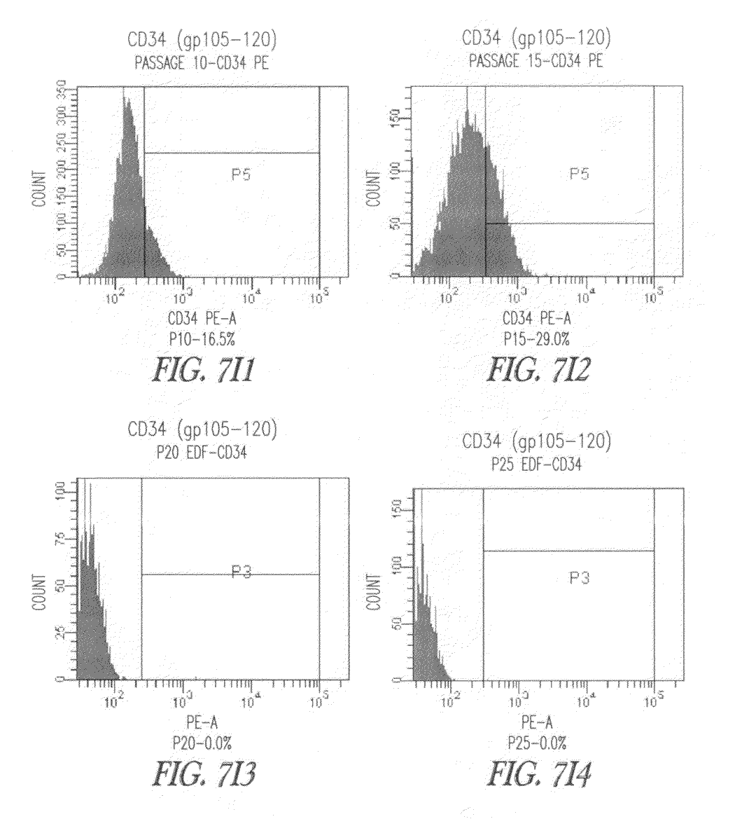

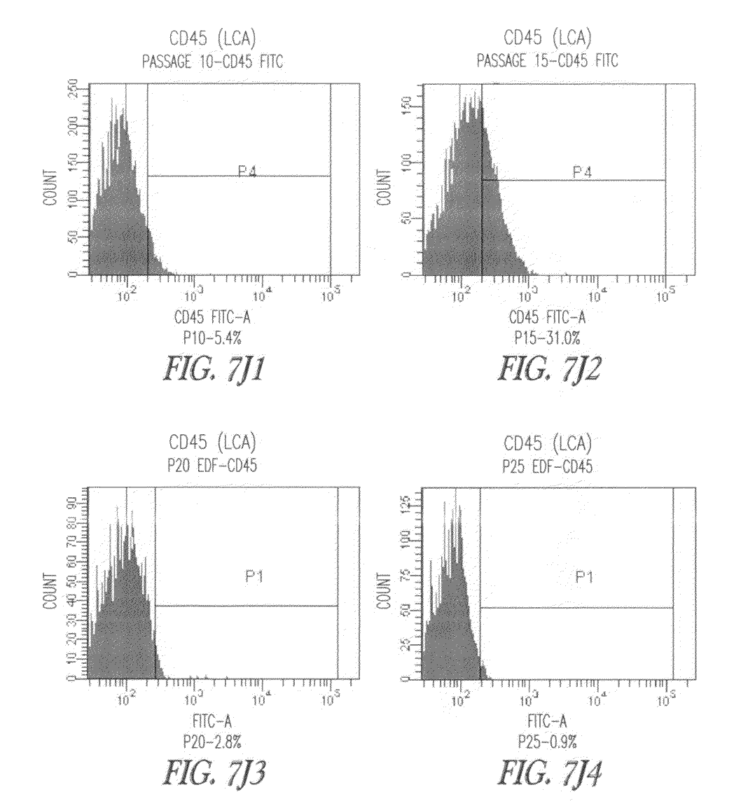

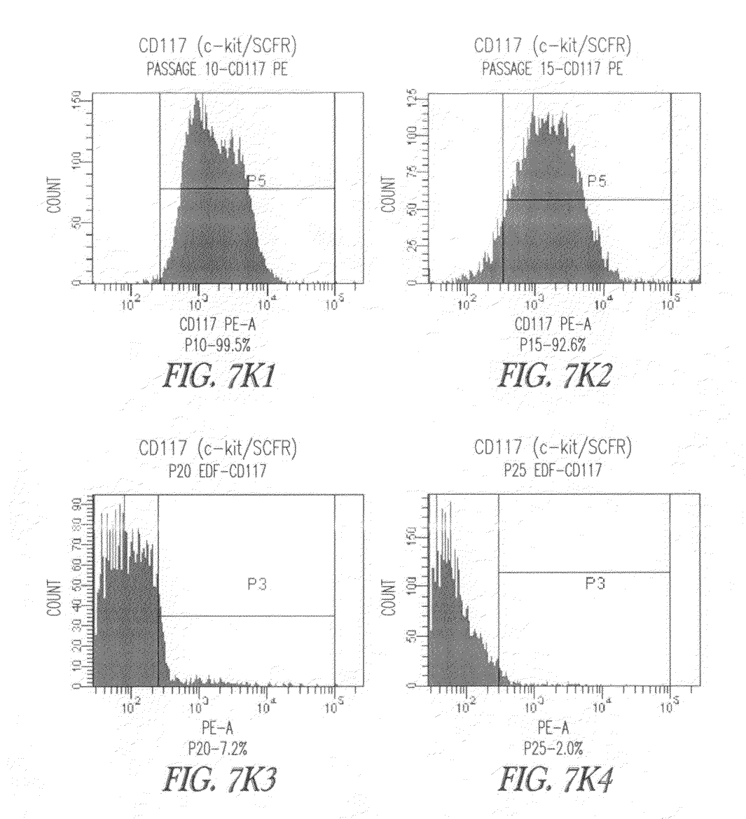

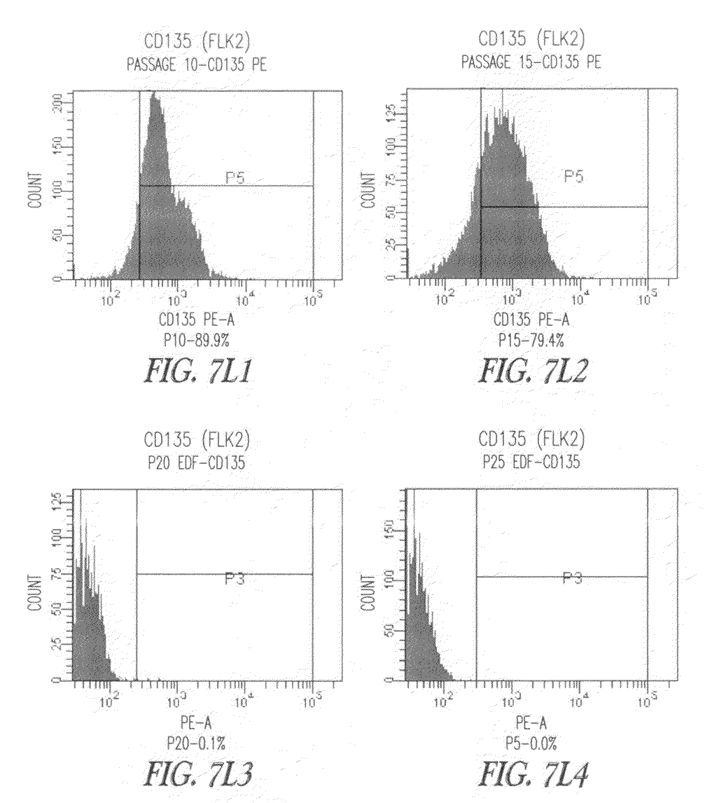

In accordance with present disclosure the human GLDF cells are characterized for specific markers in order to examine the extent of down regulation or up regulation of gene expression profile. Human GLDF cells are characterized by flow cytometry for clusters of differentiation markers (CD)/surface antigens. FIG. 7 shows the expression of cell surface markers at different passages, wherein the markers used for expression profiling of cell surface markers were CD 50, CD 106, CD 44, CD 54, CD 31, CD 105, CD 90, CD 73, CD 34, CD 45, CD 117, and CD 135. It was found that the expression level for these markers increases over passages and later on decreased. Human GLDF cells were found to be highly positive for CD90, CD44 and CD117, CD73, CD 105 whereas moderately positive for, CD106, CD50, CD54 and CD135 and negative for CD45, CD34, CD31, CD133 (See Table 4). Detailed procedure of the gene expression profile is described in the Example 2.

In one aspect the present disclosure provides undifferentiated, pluripotent and proliferative hESCs cultured on xeno-free culture system comprising human GLDF cells, wherein the hESCs are substantially free of xeno contaminants.

hESC lines co-cultured with human GLDF cells of the present disclosure maintain doubling time of at least 20-25 hours which is faster than the conventional method of using mouse embryonic feeder cells. As observed in this disclosure hESCs maintain in an undifferentiated state for long number of passages.

In one of the preferred embodiments, hESCs (HUES-7 and/or HUEC-9) have been cultured on xeno-free culture system comprising GLDF cells, following upon which they are screened for various embryonic and differentiation markers.

In accordance with the present disclosure HUES-7 and HUESC-9 lines were derived from day 5 human embryos obtained after informed consent taken from infertile patients. Institutional Ethics Committee approval was taken before obtaining embryos from the infertile patients. Only spare and supernumerary embryos were taken after the infertility treatment is over. Inner cell mass of the embryos were taken after immunosurgery and cultured on mouse embryonic feeder cells. Both the cell lines were characterized and established for prolonged culture. Method or derivation of HUES-7 and HUES-9 have been described in Example 3.

In an embodiment of the present disclosure HUES-7 cells were thawed and cultured for several passages on a Xeno-free culture system comprising feeder layer of human GLDF cells and a culture medium which further comprises about 70-90% KO-DMEM, about 10-30% human serum, about 2 mM L-glutamine, about 2% non-essential amino acids, about 0.1 mM beta-mercaptoethanol and about 4-10 nanogram per milliliter human recombinant basic fibroblast growth factor.

The details of derivation and culture of HUES cell lines are given in Example 3. FIG. 8 shows the morphology of cells of HUES-7 cell lines cultured on human GLDF cells of the present disclosure.

In yet another embodiment of the present disclosure, HUES-9 cells were cultured using xeno-free culture system as described in Example 3. FIG. 9 shows the morphology of cells of HUES-9 cell lines cultured on human GLDF cells of the present disclosure.

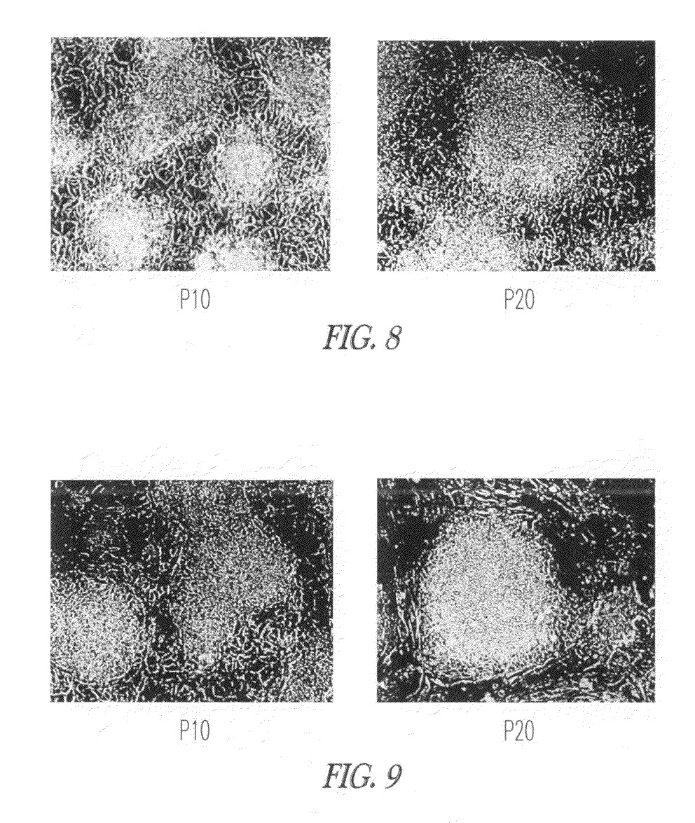

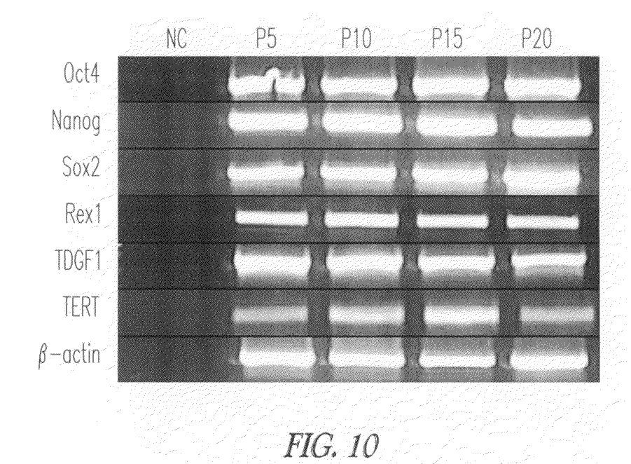

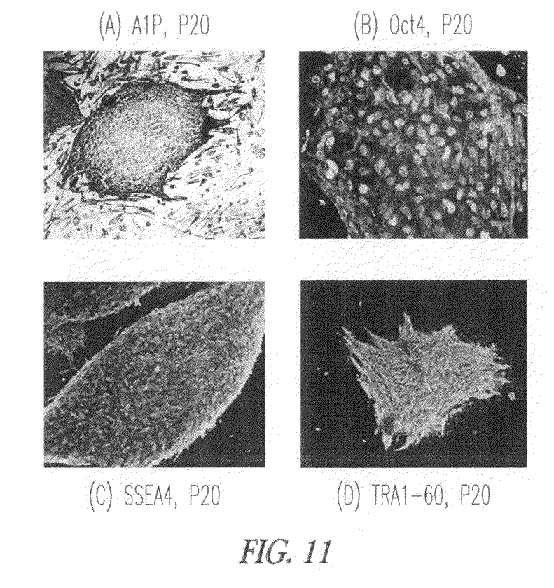

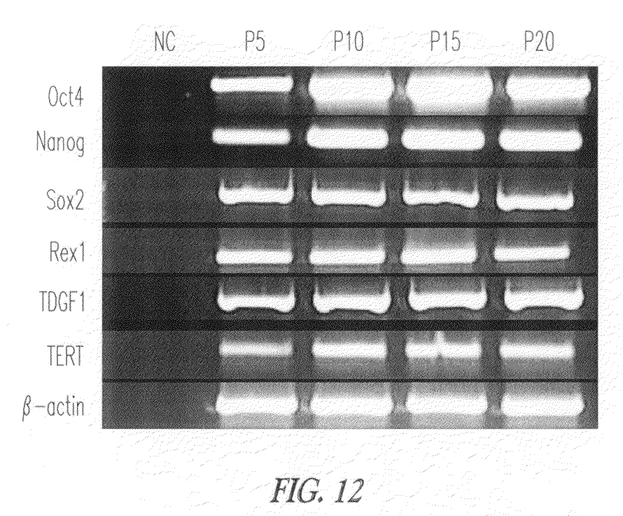

The cultured HUES-7 cells were then characterized for pluripotency by analyzing the presence of pluripotent markers. The cultured cells were found to be undifferentiated and capable of self renewable even after prolonged cultures. It was observed that the cells maintained pluripotency in prolonged, in-vitro culture conditions. FIG. 10 shows RT-PCR results for the expression profiling of pluripotent markers on HUES-7 cells, wherein the markers were OCT-4 Nanog, Sox2, Rex1, TDGF1, TERT and β-actin (Also see Table 5). GAPDH can also be used as a positive control. The marker specific primers were used in the RT-PCR reaction the nucleotide sequences for which are as shown in SEQ ID NO.: 23-32. The primer sequences used for the expression of GAPDH and OCT-4 are as shown below: and is shown in SEQ ID NO.: 37, 38 and SEQ ID NO.: 39, 40:

| | |
|---|---|
| GAPDH | |
| SEQ ID NO.: 37 - | GGGCGCCTGGTCACCAGGGCTG |
| SEQ ID NO.: 38 - | GGGGCCATCCACAGTCTTCTG |
| OCT-4 | |
| SEQ ID NO.: 39 - | CGACCATCTGCCGCTTTGAG |
| SEQ ID NO.: 40 - | CCCCCTGTCCCCCATTCCTA |

Expression profile of pluripotent markers on HUES-7 cells cultured on human GLDF cells was analyzed also by employing immunocytochemistry (See Table 6). Further, FIG. 11 shows photomicrographs illustrating the expression of embryonic stem cell markers analyzed by immunocytochemistry on HUES-7 cells cultured on GLDF cells. Expression of Alkaline phosphatase, OCT-4, SSEA-4 and TRA-1-60 was checked at Passage 20 (P20) in order to confirm the pluripotency capabilities.

In accordance with the present disclosure, the human embryonic stem cells (HUES-7 or HUES-9 as used herein) when co-cultured with the human GLDF cells, were found to remain capable of differentiating into major germ lineages as endoderm, ectoderm, and mesoderm. To confirm the differentiation of hESCs in vitro, feeder free HUES-7 cells were transferred to culture medium comprising about 80% KO-DMEM/F, about 20% KO-SR, about 1 mM L-glutamine, about 1% nonessential amino acids, about 0.1 mM β-mercaptoethanol except for bFGF and cultured continuously. At specific intervals, total RNA was isolated from cells of Embryoid bodies (EBs) using methods known in the art. The differentiation potential of cells was confirmed by performing RT-PCR for various differentiation markers on cells of EBs.

In an embodiment of the present invention, differentiation markers such as Nestin, NCAM, beta-tubulin, alpha-actinin, myosine heavy chain, brachiury, PDX, alpha fetoprotein, GATA-2, Hand-1, BMP-4 were found negative on cultured HUES-7 cells as screened by methods known in the art. Detailed procedure of the gene expression profile of HUES-7 cells cultured on GLDF cells is described in the Example 4.

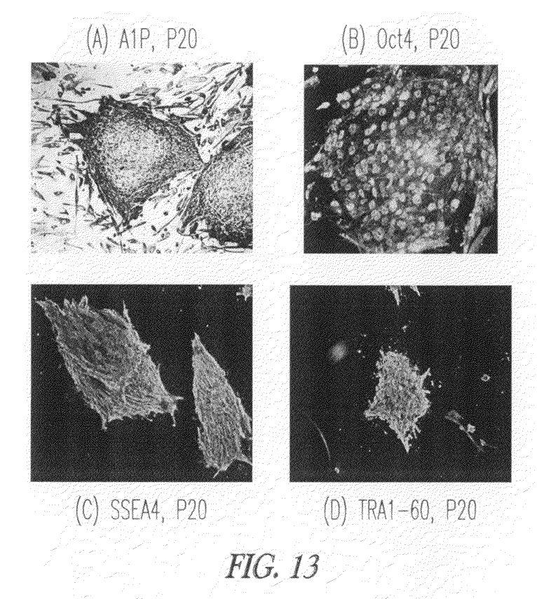

Gene expression profiling of the hESCs of HUES-9 cell line was performed using the materials and methods as discussed in example 4. FIG. 13 shows RT-PCR results illustrating the expression of various pluripotent markers on HUES-9 cultured on GLDF cells. Expression of Oct 4, Nanog, Sox 2, Rex 1, TDGF 1 and TERT was checked at different passages and was found positive.

### EXAMPLES

It should be understood that the following examples described herein are for illustrative purposes only and that various modifications or changes in light will be suggested to persons skilled in the art and are to be included within the spirit and purview of this application and the scope of the appended claims.

### Example 1

### Generation of Germ Lineage Derived Feeder Cells (GLDF Cells)

### Direct Differentiation to Obtain Embryoid Bodies

Embryoid bodies (EBs) were obtained by culturing hESCs in suspension for 7 days. hESCs were harvested by using 0.05% trypsin (invitrogen) and plated on non-tissue culture treated dishes (approximately 10⁷ cells/10 cm dish), and grown in medium for 7 days. Media comprises of KO-DMEM basal medium supplemented with 20% human serum, glutamine, 1% non-essential amino acid, beta mercaptoethanol and pen-strep. The number of EBs was determined by counting EBs in 20 different fields at a low magnification (10×) using an TE2000 microscope (Nikon). Media was changed after 3 days.

### Obtaining Germ Lineage Derived Feeder Cells (GLDF Cells)

To prepare hESC-derived feeders or the GLDF cells, EBs were plated in a T75 tissue culture flask coated with 0.1% gelatin in a GLDF media which consists of KO-DMEM supplemented with 10% KO-Serum or 10% human serum, 2 mM Glutamine, 1×10⁻⁸ M dexamethasone, 1× insulin-transferrin-selenium and 10 ng/ml epidermal growth factor. After 10 days, differentiated cells were digested with 0.05% trypsin/0.53 mM EDTA and split into two flasks (passage 1 [P1]). After 3-5 days, when cells reached 90% confluence, cells were again split to obtain Passage 2 [P2] cells. Cells of P5 and after were used as feeders and were named as GLDF feeders. For derivation and long-term culture of hESCs, cultured GLDF feeders were mitotically inactivated with 10 mg/ml mitomycin C for 2.5 h and washed three times with PBS. Mitotically inactivated GLDF were then trypsinized with trypsin-EDTA and washed twice with culture medium. The dissociated GLDF were counted and plated on gelatin-coated 35 mm dish plates at 8.0×10⁵ cells per plate. (See FIG. 1)

### Example 2

### Gene Expression Profile

### Characterization of GLDF Cells for Differentiation Markers by RT-PCR

Cells were analyzed for the differentiation markers after different passages. GLDF cells were analyzed for the expression of differentiation markers by RT-PCR. GLDF cells were positive for the expression of Nestin, NCAM, β-III tubulin, GATA2, GATA-4, BMP2, BMP4, Hand1, Vimentin, CK18, CK19, NF heavy chain, NF light chain and GFAP.

RNA extractions were carried out with the RNeasy mini kit. GLDF were vortexed for 1 min to shear genomic DNA before loading onto the columns, and then eluted in a minimum volume of 30 μl and a maximum volume of 2×50 μl RNAse-free water. RNA obtained with this procedure was essentially free of genomic DNA. When using different extraction procedures, a DNAse I treatment, followed by phenol extraction and ethanol precipitation, was applied to remove traces of contaminating DNA.

RNA obtained from the cells was reverse transcribed in the presence of 5 mM MgCl₂, 1×PCR Buffer II, 1 mM dNTPs, 25 u MuLV Reverse Transcriptase, 1 u RNA inhibitor, 2.5 μM Random hexamers in a final reaction volume of 20 μl. Reactions were carried out at 42° C. for 30 minutes in a thermocycler, followed by a 10 minute step at 99° C., and then by cooling to 4° C. 2 μl of cDNA products were amplified with 1 unit of Taq polymerase in the buffer provided by the manufacturer which contains no MgCl₂, and in the presence of the specific primers having nucleotide sequence as shown in SEQ ID NOs.: 1-20 together with the beta-actin primers (SEQ ID NO.: 21 and SEQ ID NO.: 22) used as an internal control. The amount of dNTPs carried over from the reverse transcription reaction is fully sufficient for further amplification. A first cycle of 10 minutes at 95° C., 45 seconds at 65° C. and 1 minute at 72° C. was followed by 45 seconds at 95° C., 45 seconds at 65° C. and 1 minute at 72° C. for 30 cycles. The conditions were chosen so that none of the RNAs analyzed reached a plateau at the end of the amplification protocol, i.e. they were in the exponential phase of amplification, and that the two sets of primers used in each reaction did not compete with each other. Each set of reactions always included a no-sample negative control.

The PCR products were loaded onto ethidium bromide stained 1 to 2% (depending on the size of the amplification products) agarose gels in TBE. A 100 bp DNA ladder molecular weight marker was run on every gel to confirm expected molecular weight of the amplification product.

Images of the RT-PCR ethidium bromide-stained agarose gels were acquired with a gel documentation system and quantification of the bands was performed. Band intensity was expressed as relative absorbance units (See FIG. 2). The ratio between the sample RNA to be determined and control (Beta-Actin) was calculated to normalize for initial variations in sample concentration and as a control for reaction efficiency. Mean and standard deviation of all experiments performed were calculated after normalization to beta-Actin. Results are provided in Table 1. (Refer FIG. 2)

TABLE 1
Analysis of Markers on GLDF cells

| Markers | Results |
|---|---|
| Vimentin | Positive |
| Nestin | Positive |
| NF light chain | Positive |
| NCAM | Positive |
| GFAP | Positive |
| β-III tubulin | Positive |
| GATA2 | Positive |
| GATA-4 | Positive |
| BMP2 | Positive |
| BMP4 | Positive |
| Hand1 | Positive |

### Characterization of GLDF Cells for Pluripotent Markers by RT-PCR

Cells were analyzed for the pluripotent markers after passage 4. GLDF cells were analyzed for the expression of pluripotent markers by RT-PCR. GLDF cells were negative for NANOG, SOX-2, REX-1, TDGF-1 and TERT. This clearly showed that GLDF cells lost embryonic like properties and become differentiated cells.

RT-PCR reaction was carried out as described above. The reaction was carried out in the presence of the specific primers having nucleotide sequence as shown in SEQ ID NOs.: 23-32 together with the beta-actin primers (SEQ ID NO.: 21 and SEQ ID NO.: 22) used as an internal control.

Images of the RT-PCR ethidium bromide-stained agarose gels were acquired with a gel documentation system (See FIG. 3) and quantification of the bands was performed.

TABLE 2
Analysis of Pluripotent Markers on GLDF cells (By RT-PCR)

| Pluripotent/Stemness Markers | Results |
|---|---|
| β-actin control | Positive |
| Nanog | Negative |
| Sox2 | Negative |
| Rex1 | Negative |
| TDGF1 | Negative |
| TERT | Negative |

### Characterization of GLDF Cells for Fibroblast Markers by RT-PCR

Similarly characterization of GLDF Cells for the expression of fibroblast markers was carried out using RT-PCR. The markers considered for characterization were Vimentin, P4Hβ and bFGF.

RT-PCR reaction was carried out as described above. The reaction was carried out in the presence of the specific primers for Vimentin and P4Hβ (SEQ ID NOs: 17, 18, 33 and 36) together with the beta-actin primers (SEQ ID NOs 21 and 22). Expression of beta-actin was again used as an internal control.

Images of the RT-PCR ethidium bromide-stained agarose gels were acquired with a gel documentation system (See FIGS. 4 and 5) and quantification of the bands was performed.

TABLE 3
Analysis of fibroblast markers on GLDF cells (By RT PCR)

| Fibroblast Markers | Results |
|---|---|
| Vimentin | Positive |
| P4Hβ | Positive |
| bFGF (217 bp) | Positive |

### Characterization of GLDF Cells by Immunocytochemistry

GLDF cells were fixed in 4% paraformaldehyde in phosphate buffered saline, 0.05% Triton X-100 for 30 minutes at room temperature and incubated with primary antibodies overnight at 4° C. Fluorescein isothiocyanate (FITC)-conjugated secondary antibodies (1:100); antibodies against Vimentin, Nestin and P4HB were used for the expression profiling. The specificity of each antibody was verified by negative controls included in each experiment. The slides were analyzed using inverted microscope. (See FIG. 6)

### Characterization of GLDF Cells for Differential Markers by Flow Cytometry

Characterization of cell surface cluster differentiation (CD) markers on GLDF cells to aid in analyzing the expression of cell surface markers was done. Flow cytometry showed cell populations positive for CD44, CD50, CD54, CD73, CD90, CD105, CD106, CD117 and CD135, and negative for CD31, CD34, CD45, CD133.

Aliquots of GLDF cells were allowed to expand at 37° C. and 95% air/5% CO2 humidified environment. After expansion, cells were dissociated with 0.05% trypsin-EDTA and re-suspended in buffer. The cells were then centrifuged and re-suspended in wash buffer at a concentration of 1×10⁶ cells/ml. Wash buffer consisted of phosphate buffer supplemented with 1% (v/v) FBS and 1% (w/v) sodium azide. Cell viability was >98% by the Trypan blue exclusion method. 100 μl of cell preparation 1×10⁵ were stained with saturating concentrations of fluorescein isothiocyanate-(FITC), phycoerythrin-(PE), conjugated markers and isotype matched controls. Briefly, cells were incubated in the dark for 30 min. at 4° C. After incubation, cells were washed three times with wash buffer and resuspended in 0.5 ml of wash buffer for analysis on the flow cytometer. Flow cytometry was performed on a LSR-II. Cells were identified by light scatter. Logarithmic fluorescence was evaluated (4 decade, 1024 channel scale) on 10,000 gated events. Analysis was performed using software known in the art and the presence or absence of each antigen was determined by comparison to the appropriate isotype control. An antigenic event was observed when the fluorescence was greater than 25% above its isotype control. Statistical analysis was performed on the pooled flow cytometric data from the three mesenchymal stem cell lines. Thus, a sample size of three was used for each CD marker. A mean value above 1000 cells was considered positive for any CD marker. Results are given in Table-6. (Also see FIG. 7)

TABLE 4
Analysis of Cluster of Differential Markers
on GLDF cells (By flow cytometry)

| Differential Markers | Results | Percentage |
|---|---|---|
| CD90 | Positive | 92.5% |
| CD105 | Positive | 96.5% |
| CD73 | Positive | 73.4% |
| CD45 | Negative | 31.0% |
| CD34 | Negative | 29.0% |
| CD44 | Positive | 82.3% |
| CD106 | Positive | 61.6% |
| CD31 | Negative | 11.7% |
| CD50 | Negative | 27.7% |
| CD54 | Positive | 92.6% |
| CD133 | Negative | 5.82% |
| CD117 | Positive | 92.6% |
| CD135 | Positive | 79.4% |

### Karyotyping:

It has been reported that karyotype instability can sometimes be observed with long-term passages of cells. In order to determine the karyotypic instability, karyotyping of the GLDF cells was done at different passages, preferably after every 10 passages. GLDF cells were grown in 60 mm plate on high density. Colcemid solution was added on the following day directly into the plate at the final concentration of 0.02 μg/ml. Cells were incubated for 2 hours at 37° C. and 5% CO₂. Culture media containing colcemid was removed after the incubation was over and cells were dissociated with 0.05% trypsin free from EDTA. Cells were transferred into 15 ml tube and 10 ml FBS in DMEM-F-12 was added. Cells were washed by centrifuging at 1000 rpm for 5 minutes at room temperature. Supernatant was removed and re-suspend the pellet in 2 ml of warm hypotonic solution. Cells were mixed properly and incubated in a water bath at 37° C. for 30 minutes. 0.5 ml of fixative is added drop-wise with swirling. Cells were centrifuged again at 1000 rpm for 5 minutes at room temperature. Supernatant was aspirated and 1 ml of fixative was added drop-wise while swirling the cells. This was done at least 2 times.

To make the spread, surface of the slide is humidified by application of warm breath whilst holding the slide at a 45° angle. One drop of the suspended cells is carefully dropped from the height of approximately 0.5 meter using Pasteur pipette onto the top surface of the slide and it was allowed to air dry.

Slide was stained with freshly made Leishman's stain for 8 minutes and was rinsed in running water for 1 minute and air dried. Cells were mounted with coverslip using depex.

Karyotyping of GLDF cells maintained in culture until passage 25 was found to be normal

### Example 3

### Culture and Propagation of Human Embryonic Stem Cells Using GLDF Cells

### Derivation of Human Embryonic Stem Cell Lines (HUES-7 and HUES-9)

Human embryos were produced by the ART Center, Manipal Hospital, Bangalore. Surplus embryos were used for hESC derivation with informed consent. The procedure to derive hESCs from surplus embryos was in accordance with the Guidelines of Indian Council of Medical Research (ICMR) and approved by the Ethics Committee of Manipal Hospital.

Zona pellucida of the blastocyst was removed with 0.5% pronase. Inner cell mass was isolated manually and cultured on Mit-C treated GLDF feeder cells prepared as described above. The culture medium consisted of 78% KO-DMEM/F, 20% KO-SR, 2 mM L-glutamine, 1% nonessential amino acids, 0.1 mM β-mercaptoethanol, and 4 ng/ml bFGF. The medium was changed every day. Ten to 14 days after initial plating, colonies with typical hESCs morphology appeared. These colonies were dissociated mechanically and transferred onto a fresh dish with human GLDF cells.

### Culture and Propagation of Human Embryonic Stem Cell Lines

HUES-7 and HUES-9 cells has been cultured using GLDF cells. However, hESCs obtained from various sources can be cultured and propagated using GLDF cells. hESCs (HUES-7 or HUES-9) were trypsinized with trypsin-EDTA and washed twice with media and transferred to culture dishes preplated with Human GLDF cells. Long-term culture of hESCs was performed by passaging hESCs every 5-6 days using trypsin in combination with manual dissociation. hESCs were cryopreserved in freezing media consisting of 90% KO-SR and 10% dimethylsulfoxide.

To determine population doubling (PD) time, cell numbers in five selected independent colonies were counted under an inverted microscope. Data collected on days 1 and 2 (with 36 hours apart) were used to calculate PD values: PD=log 2, in which N1 and N2 are the cell numbers of selected colonies counted on day 1 and day 2, respectively.

See FIGS. 8 and 9 for the morphology of HUES-7 and HUES-9 cells cultured on human GLDF cells.

### Example 4

### Gene Expression Profiling of hESCs

Characterization of hES cells for pluripotent markers by RT-PCR:

HUES-7 cells were analyzed for the expression of pluripotent markers at passage 4 by RT-PCR and were positive for OCT-4, Nanog, as compared with the expression of Beta-actin marker which was used as positive control.

RT-PCR reaction was carried out as described above. The reaction was carried out in the presence of the specific primers. Primer sequences for OCT-4, Nanog, Rex-1, TDGF, TERT, and SOX-2 are as shown in SEQ ID NOs.: 23-32 and SEQ ID NOs.: 39 and 40. The expression of GAPDH marker can also be used as an internal control, the primer sequences in that case is as set forth in SEQ ID NO.: 37 and 38.

Images of the RT-PCR ethidium bromide-stained agarose gels were acquired with a gel documentation system (See FIG. 10) and quantification of the bands was performed.

TABLE 5
Primer Sequences Used in PCR:

| Primers | Size | Results |
|---|---|---|
| OCT-4 | 572 | Positive |
| Nanog | 262 | Positive |
| Rex-1 | 303 | Positive |
| TDGF1 | 498 | Positive |
| SOX2 | 448 | Positive |
| TERT | 602 | Positive |
| GAPDH | 564 | Positive control |

### Characterization of Human Embryonic Stem Cell Lines by Immunocytochemistry

Immunocytochemistry was performed as explained above in Example 2. Fluorescein isothiocyanate (FITC)-conjugated secondary antibodies (1:100) against SSEA-1 (1:100), SSEA-3 (1:200), SSEA-4 (1:200), TRA-1-60 (1:100), and TRA-1-81 (1:100), Sox-2 and alkaline phosphatase were used. The results are given below in Table 8 (Also see FIG. 11)

TABLE 6
Analysis of Markers on hESCs (By Immunocytochemistry)

| Markers | Results |
|---|---|
| SSEA-1 | Negative |
| SSEA-3 | Positive |
| SSEA-4 | Positive |
| SOX-2 | Positive |
| Alkaline Phosphatase | Positive |
| TRA-1-60 | Positive |
| TRa-1-81 | Positive |

The above analysis indicates that the GLDF cells are a suitable medium for derivation, culture and propagation of hESCs for several passages in undifferentiated state. The hESCs co-cultured with human GLDF cells maintain their pluripotency and remain capable of differentiating into cells of germ lineages like mesoderm, endoderm and ectoderm.

### BIBLIOGRAPHY

Amit M, Margulets V, Segev H et al. Human feeder layers for human embryonic stem cells. Biol Reprod 2003; 68:2150-2156.
Amit M, Shariki C, Margulets V et al. Feeder layer- and serum-free culture of human embryonic stem cells. Biol Reprod 2004; 70:837-845.
Amit M, Winkler M E, Menke S, Bruning E, Buscher K, Denner J, Haverich A, Itskovitz-Eldor J, Martin U. No evidence for infection of human embryonic stem cells by feeder cell-derived murine leukemia virus. Stem Cell 2005. 23:761-771.
Cheng L, Hammond H, Zhaohui Y et al. Human adult marrow cells support prolonged expansion of human embryonic stem cells in culture. Stem Cells 2003; 21:131-142.
Evans M J, Kaufman M H. Establishment in culture of pluripotential cells from mouse embryos. Nature. 1981 Jul. 9; 292(5819):154-6.
Graves K H, Moreadith R W. Derivation and characterization of putative pluripotential embryonic stem cells from preimplantation rabbit embryos. Mol Reprod Dev. 1993 December; 36(4):424-33
Heins N, Englund M C, Sjoblom C, Dahl U, Tonning A, Bergh C, Lindahl A, Hanson C, Semb H. Derivation, characterization, and differentiation of human embryonic stem cells. Stem Cells. 2004; 22(3):367-76.
Iannaccone P M, Taborn G U, Garton R L, Caplice M D, Brenin D R. Pluripotent embryonic stem cells from the rat are capable of producing chimeras. Dev Biol. 1994 May; 163(1):288-92.
Lee J B, Song J M, Lee J E, Park J H, Kim S J, Kang S M, Kwon J N, Kim M K, Roh S, Yoon H S. Available human feeder cells for the maintenance of human embryonic stem cells. Reproduction 2004. 128: 727-735
Martin G R. Isolation of a pluripotent cell line from early mouse embryos cultured in medium conditioned by teratocarcinoma stem cells. Proc Natl Acad Sci USA. 1981 December; 78(12):7634-8.
Martin M J, Muotri A, Gage F, Varki A. Human embryonic stem cell expresses an immunogenic non-human sialic acid. Nat. Med. 2005. 11: 228-232.
Notarianni E, Galli C, Laurie S, Moor R M, Evans M J. Derivation of pluripotent, embryonic cell lines from the pig and sheep. Department of Genetics, University of Cambridge, UK. J Reprod Fertil Suppl. 1991; 43:255-60.
Reubinoff B E, Pera M F, Vajta G et al. Effective cryopreservation of human embryonic stem cells by the open pulled straw vitrification method. Hum Reprod 2001; 16:2187-2194.
Richards M, Tan S, Fong C-Y et al. Comparative evaluation of various human feeders for prolonged undifferentiated growth of human embryonic stem cells. Stem Cells 2003; 21: 546-556.
Shamblott M J, Axelman J, Wang S, Bugg E M, Littlefield J W, Donovan P J, Blumenthal P D, Huggins G R, Gearhart J D. Derivation of pluripotent stem cells from cultured human primordial germ cells. Proc Natl Acad Sci USA. 1998 Nov. 10; 95(23):13726-31. Erratum in: Proc Natl Acad Sci USA 1999 Feb. 2; 96(3):1162.
Thomson J A, Kalishman J, Golos T G, Durning M, Harris C P, Becker R A, Hearn J P. Isolation of a primate embryonic stem cell line. Proc Natl Acad Sci USA. 1995 Aug. 15; 92(17):7844-8.
Thomson, J. A., Itskovitz-Eldor, J., Shapiro, S., Waknitz, M. A., Sweirgiel, J. J., Marshall, V. S., et al. (1998). Embryonic stem cell lines derived from human blastocysts. Science, 282, 1145-1147.
Xu C, Inokuma M S, Denham J et al. Feeder-free growth of undifferentiated human embryonic stem cells. Nat Biotechnol 2001; 19: 971-974.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method of generating human germ lineage derived feeder cells (GLDF cells), said method comprising:
a. culturing human embryonic stem cells (hESC) on growth medium to obtain germ lineage cells;
b. culturing said germ lineage cells on GLDF medium to obtain fibroblast like cells; and
c. treating said fibroblast like cells to generate human GLDF cells.

2. The method as claimed in claim 1, wherein the growth medium comprises KO-DMEM, serum replacement, about 1 mM glutamine, about 1% nonessential amino acids (NEAA), about 0.1 mM β-mercaptoethanol, and an antibiotic.

3. The method as claimed in claim 1, wherein the GLDF medium comprises of KO-DMEM, growth factors, serum supplement or a combination thereof.

4. The method as claimed in claim 3, wherein the growth factors are selected from a group consisting of transforming growth factor-β-1 (TGF-β-1), epidermal growth factor (EGF), brain derived neurotrophic factor (BDNF), platelet derived growth factor (PDGF), Insulin, selenite, transferrin, Activin-A, Activin-B, Acidic FGF, human Insulin growth factor (IGF), Keratenocyte growth factor (KGF), stem cell factor (SCF), bone morphogenic protein (BMP4), hepatocyte growth factor (HGF), nerve growth factor (NGF) and a combination thereof.

5. The method as claimed in claim 1, wherein the GLDF medium comprises KO-DMEM, about 10% KO-Serum, about 1× Insulin-transferrin-selinium, about 1×10⁻⁸ dexamethasone, about 1% glutamine, antibiotic, about 10 ng/ml epidermal growth factor (EGF) or a combination thereof.

6. The method as claimed in claim 4, wherein the TGF-β-1 is provided at a concentration of about 1-20 ηg/ml.

7. The method as claimed in claim 4, wherein the EGF is provided at the concentration of about 1-20 ng/ml.

8. The method as claimed in claim 4, wherein the activin A is provided at the concentration of about 5-100 ng/ml.

9. The method as claimed in claim 4, wherein the activin B is provided at the concentration of about 5-100 ng/ml.

10. The method as claimed in claim 4, wherein the acidic FGF is provided at the concentration of about 1-20 ng/ml.

11. The method as claimed in claim 4, wherein the BDNF is provided at the concentration of about 1-20 ng/ml.

12. The method as claimed in claim 4, wherein the PDGF is provided at the concentration of about 1-20 ηg/ml.

13. The method as claimed in claim 4, wherein the IGF is provided at the concentration of about 2-20 ng/ml.

14. The method as claimed in claim 4, wherein the KGF is provided at the concentration of about 10-50 ng/ml.

15. The method as claimed in claim 4, wherein the SCF is provided at the concentration of about 5-20 ng/ml.

16. The method as claimed in claim 4, wherein the BMP4 is provided at the concentration of 5-20 ng/ml.

17. The method as claimed in claim 4, wherein the HGF is provided at the concentration of about 10-20 ng/ml.

18. The method as claimed in claim 4, wherein the NGF is provided at the concentration of about 20-100 ng/ml.

19. The method as claimed in claim 1, wherein said fibroblast like cells were treated with mitomycin.

20. Human GLDF cells for culturing human embryonic stem cells (hESCs), wherein the GLDF cells are generated by the method as claimed in claim 1.

21. The human GLDF cells as claimed in claim 20, wherein the cells are fibroblast-like cells.

22. The human GLDF cells as claimed in claim 20, wherein the cells are negative for the expression of pluripotent markers selected from the group consisting of OCT-4, NANOG, REX-1, TDGF, SOX-2, and TERT.

23. The human GLDF cells as claimed in claim 20, wherein the cells are positive for the fibroblast phenotypic marker P4HB and mesenchymal stem cell marker Vimentin.

24. The human GLDF cells as claimed in claim 20, wherein cells are positive for mesenchymal stem cell markers CD73, CD105, CD90, CD44.

25. The human GLDF cells as claimed in claim 20, wherein the cells are positive for the expression of differentiation markers selected from the group of NCAM, β-III tubulin, GATA2, Hand 1, BMP4, Vimentin, CK18, Nestin, NF heavy chain, GFAP, and CK19.
